(19) United States
(12) Patent Application Publication
(10) Pub. No.: US 2016/0310187 A1
(43) Pub. Date: Oct. 27, 2016

(54) SELF-TAPPING, TENSION ABSORBING BONE SCREW

(71) Applicant: RITA LEIBINGER GMBH & CO. KG, Neuhausen ob Eck (DE)

(72) Inventors: Rita Leibinger, Mullheim (DE); Enrico Wirth, Donaueschingen (DE)

(73) Assignee: Rita Leibinger GmbH & Co. KG, Neuhausen ob Eck (DE)

(21) Appl. No.: 15/102,373

(22) PCT Filed: Dec. 19, 2014
(86) PCT No.: PCT/EP2014/078669

§ 371 Date: Jun. 7, 2016

(30) Foreign Application Priority Data
Dec. 19, 2013 (DE) 102013226704.5

(51) Int. Cl. A61B 17/86; A61B 17/70

(52) U.S. Cl. CPC A61B 17/863; A61B 17/7032; A61B 17/7035; A61B 17/8635; A61B 17/864

(57) ABSTRACT

A self-tapping, self-drilling, cannulated bone screw having a channel chamber. This screw is characterized in that it includes a centering tip which forms the front end of the bone screw.

BACKGROUND

The invention relates to a self-cutting and self-drilling cannulated bone screw with a channel chamber. Bone screws of this kind are used, for example, in osteosynthesis of complicated bone fractures. Self-cutting has the meaning here that there is no need for preliminary cutting of a thread in the bone structure that is to be treated. Self-drilling is to be understood as meaning that the bone screw, during insertion into the bone structure, is able to drill itself in, and there is therefore no need for preliminary drilling.

### SUMMARY

The object of the present invention is to make available a bone screw which simplifies the treatment of bone fractures and in particular reduces the number of work steps needed for insertion of the bone screw.

In a bone screw of the aforementioned type, this object is achieved by the fact that it comprises a centering tip, which forms a front end of the bone screw. This permits simple positioning and precise insertion of the bone screw according to the invention into the bone structure that is to be treated.

It has proven particularly advantageous in this case if at least one cutting device is arranged in the area of the centering tip. Thus, the cutting procedure begins directly after the bone screw has been applied with precise positioning, which reduces the risk of the bone screw slipping from the desired position. Moreover, only a short uncut depth of penetration of the centering tip into the bone is needed, which reduces the force that has to be applied by the operator and likewise reduces the risk of stress-induced damage to the bone. Overall, the time needed to insert the bone screw is reduced. The overall duration of the operation is shortened in this way, which has an advantageous effect on the wound-healing process. The channel chamber can extend in a rectilinear or spiral shape in this case.

A development of the bone screw according to the invention is characterized in that the cutting device is assigned a preferably substantially axial access opening to the channel chamber. In this way, bone material, which is detached from the bone structure to be treated during the self-drilling and self-cutting insertion of the bone screw, is introduced directly into the channel chamber. If the access opening is at least substantially axial, the bone material is additionally transported away along the shortest path and without any appreciable change of direction, which reduces the risk of a blockage. Moreover, this promotes the fusion of the bone screw according to the invention to the bone structure that is to be treated. By virtue of the self-cutting and self-drilling function of the bone screw, in combination with the removed bone material being carried away into the channel chamber, the occurrence of stress-induced damage on the bone structure to be treated is reduced.

It has proven particularly advantageous if the bone screw according to the invention has at least two cutting devices distributed preferably uniformly about the circumference, and each cutting device is assigned a substantially axial access opening to the channel chamber. A plurality of cutting devices permits more effective and therefore more rapid cutting and drilling of the bone screw into the bone structure that is to be treated. The removed bone material arising on each cutting device can be conveyed to the channel chamber via the axial access openings, and this, as has been stated above, has an advantageous effect on the wound-healing process.

It is also advantageous if the channel chamber opens into a rear end face of the bone screw. In this way, the bone material carried into the channel chamber can as it were migrate into the bone screw, and excess bone material can emerge from the bone screw in the area of the rear end face. In this way, the bone material that has been cut off is collected in the channel chamber and is able to support fusion of the bone screw. This design of the bone screw also promotes the fusion of the bone screw into the bone structure that is to be treated.

It is particularly advantageous if the bone screw according to the invention is a Herbert screw. A Herbert screw is a special bone screw which is used, for example, in the osteosynthesis of fractures of the scaphoid bone. Such a bone screw is a double-threaded screw, that is to say it has an outer thread both in a front area and in a rear area. Herbert screws are generally cannulated, that is to say they are hollow. However, known Herbert screws are neither self-cutting nor self-drilling, and instead they are inserted by the use of a guide wire, also called a Kirschner wire, into a pre-drilled hole in the bone structure to be treated, in which hole a thread has been cut. The two threads in the front area and rear area of the Herbert bone screw have different pitches. The thread pitch on the front thread is greater in this case than on the rear thread, such that a rear bone fragment is drawn mechanically onto a front bone fragment during the screwing-in procedure. In this way, a pressure that favors the healing of the fracture is applied to a fracture gap lying between the front bone fragment and the rear bone fragment. This is also referred to as interfragmentary compression.

When a Herbert screw is used for osteosynthesis, for example of a damaged scaphoid bone, the broken scaphoid bone is first of all positioned and, if necessary, fixed, and then the Herbert screw is inserted. In conventional Herbert screws, this insertion requires a first step of drilling a hole. In a second step, this hole is provided with a thread. Only in a third step can the Herbert screw be inserted. When using the bone screw according to the invention, the first two steps are dispensed with, and the bone screw according to the invention can be inserted directly and therefore very quickly into the bone structure that is to be treated.

It has proven particularly advantageous if the bone screw, at its rear end, has a screw head with an abutment face preferably in the form of a radially outwardly extending collar. In this way, the interfragmentary compression can be increased. Within the context of the present invention, however, it is also in principle conceivable to use bone screws that do not have a screw head.

It has also proven particularly advantageous if at least one substantially radial opening extends from the channel chamber to a thread turn of an outer thread present in a front area and/or in a rear area of the bone screw, preferably to a thread root. Once the bone screw has been inserted into the bone structure that is to be treated, the inner channel chamber is filled with the bone material that arises during the self-drilling and self-cutting. This bone material can fuse with the surrounding bone structure via the radial opening. In this way, the time taken for the treated fracture to heal is reduced, and the stability of the synthesis is improved.

Within the meaning of the invention, it is likewise conceivable that the bone screw has at least one recess, for example an approximately semicircular recess, in a protruding edge of a flank of an outer thread present in the front area of the bone screw, which recess forms, at least in part, a further cutting device. A further cutting device of this kind further improves the self-cutting of the bone screw that is to be inserted into the bone structure that is to be treated. In this way, moreover, there is less irritation of the bone structure that is to be treated, and there is a cleaner cutting process.

Within the meaning of the invention, it is likewise conceivable that the bone screw has at least one substantially axial through-opening in a flank of an outer thread present in a front area and/or in a rear area of the bone screw. In this way, the bone structure to be treated can grow through the thread flanks of the bone screw according to the invention, which promotes fusion of the bone screw to the bone structure. The time needed for the wound-healing process is reduced in this way, and the stability of the synthesis is further improved. Removed bone material is also able to collect in the though-openings, which leads to a reduction of stresses in the bone structure that is to be treated.

Within the meaning of the invention, a bone screw is likewise conceivable which has a through-opening in each of at least two successive flanks of at least one outer thread, wherein the through-openings are preferably in alignment with each other. This permits a stable growth of the bone screw into the bone structure that is to be treated.

According to the invention, it is also conceivable that the bone screw has an outer thread which has at least one thread channel extending inside a flank of the outer thread and preferably extending at least in part in the circumferential direction, one end of said thread channel opening into an outer face of the outer thread. It is particularly preferable here if one end of the thread channel is arranged in an additional cutting edge on the outer thread of the bone screw or if one of the cutting devices extends as far as the channel chamber. It is moreover advantageous if the thread channel substantially follows a pitch of the outer thread.

When such a bone screw with at least one thread channel is inserted into a bone structure, bone material removed from the bone structure by the additional cutting edge is carried away from the cutting site through the thread channel.

It is also advantageous if the thread channel opens with its other end into the channel chamber or into a next additional cutting edge on the outer thread. The next cutting edge is to be understood as that cutting edge which, viewed from the front end to the rear end of the bone screw, follows the previous additional cutting edge along the profile of the outer thread.

In this way, removed bone material is carried away to the channel chamber or carried from one additional cutting edge to the next additional cutting edge.

Both of the above-described types of removal of bone material from the cutting site, i.e. through the thread channels to the channel chamber and/or to the next additional cutting edge, allow the bone screw to be inserted into a bone structure without stresses being caused by bone material that is not removed during the insertion, which stresses could damage the bone structure. This is particularly advantageous if the bone screw is to be inserted into the bone structure without a separate preliminary drilling step.

The thread channel which extends at least in part inside a flank of the outer thread and preferably at least in part in the circumferential direction, and of which one end opens into an outer face of the outer thread, also constitutes an independent invention, which is also worthy of protection without the centering tip. This applies also to the indicated developments and details of said thread channel.

It is also advantageous if the bone screw comprises a receiving part for connecting the bone screw to a rod system. It is particularly advantageous here if the receiving part is connected to the bone screw in a polyaxial, uniplanar or monoaxial manner. In this way, the use of the bone screw in stiffening or fixing procedures is simplified.

According to the invention, it is also conceivable that the bone screw has a screw head, which has a locking device, preferably a head-side outer thread, for locking the bone screw at a stable angle onto a bone plate. It is particularly preferable here if the locking device is a self-cutting head-side outer thread for locking the bone screw at a stable angle onto a bone plate. This configuration has the advantage that the angle can be fixed quickly and in a manner individually adapted to the local circumstances at the operating site.

An alternative embodiment within the meaning of the invention is characterized in that the bone screw has a screw head which in part has a dome-like configuration and via which the bone screw can be connected at a variable angle to a bone plate. This permits mobility between bone screw and bone plate.

It will also be noted that the bone screw according to the invention, independently of its specific design, is preferably produced with the aid of a 3D printing method. With such a 3D printing method, highly complex three-dimensional structures can be produced, in particular for example the above-mentioned internal channels and channel chambers. A powder or another “printable or injectable” material, for example from a titanium alloy, is printed in layers by the 3D printer and fused by the use of a laser, for example. Further possible materials are ceramic, plastic, other metals, for example magnesium, etc.

Further features, details and advantages of the invention will become clear from the attached claims, from the drawing, and from the following description of several preferred embodiments of the bone screw according to the invention. An example of the present invention is explained in more detail below with reference to the attached drawings.

### BRIEF DESCRIPTION OF THE DRAWINGS

In the drawings:

FIG. 1 shows a schematic sectional view of a first embodiment of a bone screw according to the invention;

FIG. 2 shows a rear partial area of the bone screw shown in FIG. 1;

FIG. 3 shows a front partial area of the bone screw shown in FIG. 1;

FIG. 4 shows a perspective view of the bone screw shown in FIG. 1;

FIG. 5 shows a schematic side view of a further embodiment of a bone screw according to the invention;

FIG. 6 shows a section through the bone screw from FIG. 5 along the line VI-VI;

FIGS. 7A-E show different views of a further embodiment of the bone screw according to the invention with a receiving part;

FIG. 8 shows a side view of several of the bone screws shown in FIG. 7, said bone screws being connected via a rod system;

FIGS. 9A-C show different views of a further embodiment of the bone screw according to the invention;

FIGS. 10A-C show different views of a further embodiment of the bone screw according to the invention; and

FIGS. 11A-C show different views of a further embodiment of the bone screw according to the invention.

### DETAILED DESCRIPTION OF THE PREFERRED EMBODIMENTS

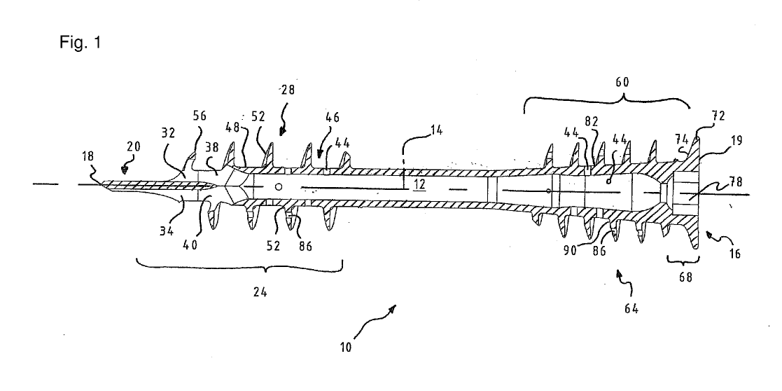

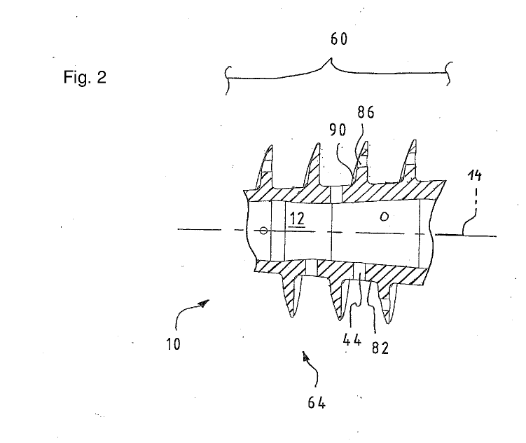

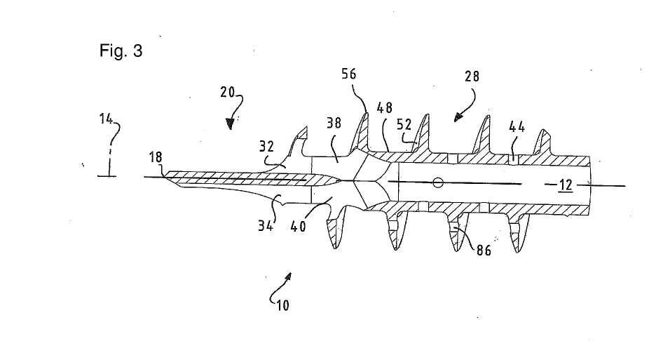

A bone screw according to the invention is designated overall by reference sign 10 in FIGS. 1 to 4. An elongated channel chamber 12, which is overall straight in the present example, extends through the inside of the bone screw. In an embodiment not shown, the channel chamber overall has a spiral shape or helical shape, or it has at least one wall with an outer wall that is spiral-shaped with respect to a longitudinal axis of the channel chamber. In the illustrated embodiment shown, the channel chamber 12 overall is in alignment with a longitudinal axis 14. The bone screw 10 extends along the longitudinal axis 14 from a rear end 16 to a front end 18. In the present illustrative embodiment, the rear end 16 is formed by a rear end face 19.

The front end 18 is formed by a centering tip 20. The centering tip 20 is integrally formed on a front area 24 of the bone screw 10. A first outer thread 28 is arranged on the bone screw 10 in the front area 24. A first cutting device 32 and a second cutting device 34 are arranged in the area of the centering tip 20. The first cutting device 32 is assigned a first access opening 38 to the channel chamber 12. The second cutting device 34 is assigned a second access opening 40 to the channel chamber 12. The first cutting device 32 and the second cutting device 34 are distributed uniformly about the circumference, i.e. they are arranged diametrically with respect to the centering tip 20. The first access opening 38 and the second access opening 40 extend axially, that is to say they extend substantially parallel overall to the longitudinal axis 14. The first access opening 38 and the second access opening 40 each extend from their assigned cutting devices 32, 34 to the channel chamber 12.

Radial openings 44 extend from the channel chamber 12 to a thread turn 46 of the first outer thread 28 arranged in the front area 24. In this illustrated embodiment, the radial openings 44 shown open from the channel chamber 12 into the thread root 48 of the thread turn 46. However, it is also conceivable that, proceeding from the channel chamber 12, they open into a front or rear face of a flank 52 of the first outer thread 28. The flank 52 of the first outer thread 28 has a protruding edge 56. The latter also plays a role as explained below.

Seen from the front end 18 of the bone screw 10 along the longitudinal axis 14 to the rear end 16, the bone screw 10 has a rear area 60 in front of the rear end 16. A second outer thread 64 is arranged in this rear area 60. The pitch of the outer thread 64 is greater than that of the first thread 28, such that a “Herbert screw” is formed. A screw head 68 is integrally connected to the rear area 60. The screw head 68 has a radially outwardly extending collar 72 which, in the operational position, forms an abutment face 74 toward the bone. A hexagon socket 78 is present on the screw head 72 and constitutes a tool attachment site. Within the meaning of the present invention, however, other types of tool attachment sites are also possible, for example a slot, cross slot, Torx, or similar.

In the rear area 60, radial openings 44 are likewise arranged which extend from the channel chamber 12 to a thread root 82 of the rear outer thread 64. Axial through-openings 86, i.e. extending parallel to the longitudinal axis 14 and in alignment with one another, extend through the flanks 52 of the first outer thread 28 and also through flanks 90 of the second outer thread 64.

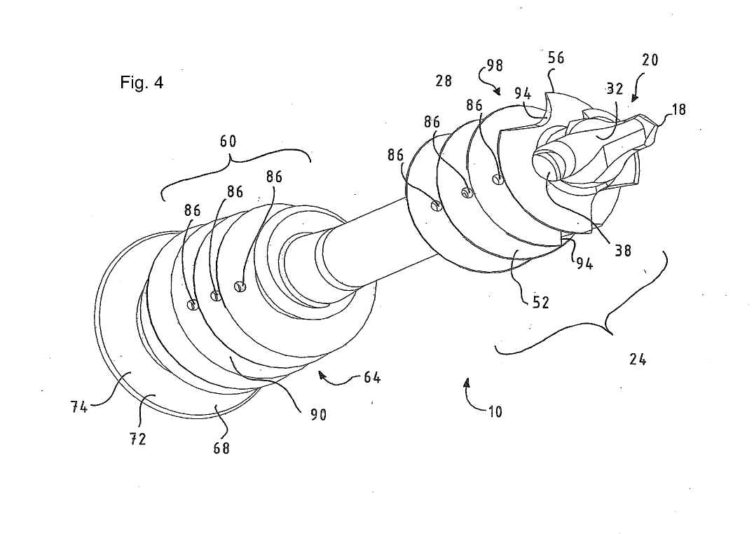

As can be seen in particular from FIG. 4, the protruding edge 56 of the flank 52 of the first outer thread 28 present in the front area 24 of the bone screw 10 has approximately semicircular recesses 94. The recesses 94 form an additional cutting edge 98.

When the bone screw 10 shown in FIG. 1 is used for osteosynthesis, the bone screw 10 is positioned using the centering tip 20. By the use of a suitable tool, the bone screw 10 is then rotated via the tool attachment site, configured as a hexagon socket 78 in the present embodiment. Due to the rotation, the first cutting device 32 and the second cutting device 34 remove bone material from the bone structure that is to be treated. This bone material is guided through the first axial access opening 38 and the second axial access opening 40 into the channel chamber 12.

Once the bone screw 10 has been inserted fully into the bone structure that is to be treated, the channel chamber 12 is substantially full of removed bone material. This bone material is in part guided outward through the radial openings 44. In this way, it comes into contact with the surrounding bone structure. As the bone screw 10 remains in the bone structure to be treated, this promotes fusion of the bone screw 10 to the surrounding bone structure. By way of the axially extending through-openings 86, the bone structure is also able to grow through the flanks 52, 90 of the outer threads 28, 64. An advantage of the bone screw 10 according to the invention is that, by virtue of the centering tip 20 in combination with the cutting devices 32, 34, there is no need for preliminary drilling of a hole and preliminary cutting of a thread in the bone structure. Moreover, no guide wire or Kirschner wire is needed to insert the bone screw 10 into the bone structure that is to be treated. By means of the centering tip 20, the bone screw 10 can be positioned precisely and inserted into the bone structure. This shortens the operating time, as a result of which the wound-healing process is promoted. Overall, therefore, the use of the bone screw 10 has a positive effect on the course of treatment.

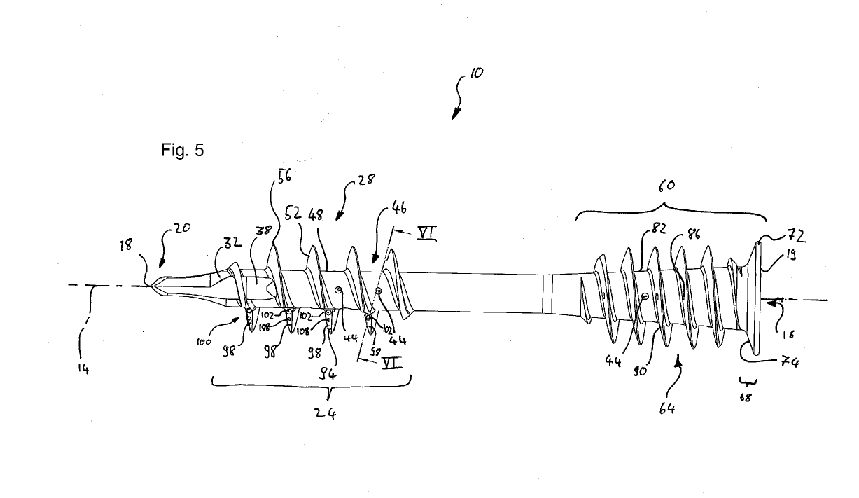

FIG. 5 shows a further embodiment of the bone screw 10 according to the invention. The bone screw 10 shown in FIG. 5 has several additional cutting edges 98 in the lower outer thread 28 in FIG. 5. The additional cutting edges 98 are formed by right-angled recesses 100 (seen in the direction of the longitudinal axis 14) in the flank 52 of the outer thread 28. Embodiments not shown can also have other recesses as such which, seen in the longitudinal axis, have right-angled and rectilinear boundary edges. For example, angles of less than or more than 90° are also conceivable, and also convexly or concavely curved boundary edges.

On the additional cutting edges 98 belonging to the outside of the outer thread 28, openings are visible which constitute one end of thread channels 102 and 108 and which are circular in the present example. Within the meaning of the invention, however, other shapes of the openings are also possible, for example triangular, square, star-shaped or similar. The thread channels 102 here extend from said openings as far as the channel chamber 12. The thread channels 102 extend inside the flank 52 of the outer thread 28 and approximately in the circumferential direction, but spiraling inward in the direction of the channel chamber 12.

The thread channels 108 each extend from said openings in an additional cutting edge 98 to a next additional cutting edge 98 as seen in the circumferential direction. Thus, the next cutting device 98 is in each case to be understood as the cutting device 98 following along the profile of the flank 52 of the outer thread 28, as seen from the front end 18 to the rear end 16 of the bone screw 10.

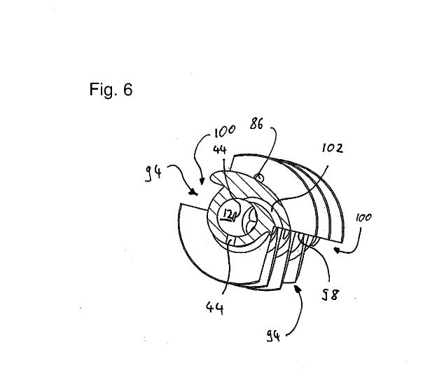

The profile of the thread channels 102 can be seen particularly clearly from the view in FIG. 6, which shows a section through the bone screw 10 in FIG. 5 along the line VI-VI. The thread channel 102 shown extends approximately in the circumferential direction and substantially follows the profile of the flank 52 of the first outer thread 28. The thread channel 102 thus follows a pitch of the first outer thread 28 and extends in a gentle spiral shape radially inward in the direction of the channel chamber 12. The profile of the thread channels 108 (not visible in FIG. 6) likewise substantially follows the profile of the outer thread 28. However, the thread channels 108 do not extend toward the channel chamber 12 like the thread channels 102, and instead they extend at an approximately constant distance from the longitudinal axis 14 of the bone screw 10. The bone screw 10 shown in FIGS. 5 and 6 can be produced, for example, with the aid of a 3D printing method, in which ceramic, plastic or metal, for example titanium or magnesium, is applied as powder or the like in layers and is fused by the use of a laser, for example.

When the bone screw 10 shown in FIGS. 5 and 6 is inserted into a bone structure, bone material removed from the bone structure by the additional cutting edges 98 is conveyed from the cutting site to the channel chamber 12 by way of the thread channels 102 that extend from the outer thread 28 as far as the channel chamber 12.

The thread channels 108, which extend from an additional cutting edge 98 to a next additional cutting edge 98, carry removed bone material from an additional cutting edge 98 to the next cutting edge 98.

Both of the above-described types of removal of bone material, from the cutting site through the thread channels 102 to the channel chamber 12 and through the chip-conveying openings 108 to the next additional cutting edge 98, allow the bone screw 10 to be inserted into a bone structure without stresses occurring during the insertion that could damage the bone structure. This is particularly advantageous if the bone screw 10 is to be inserted into the bone structure without a separate preliminary drilling step.

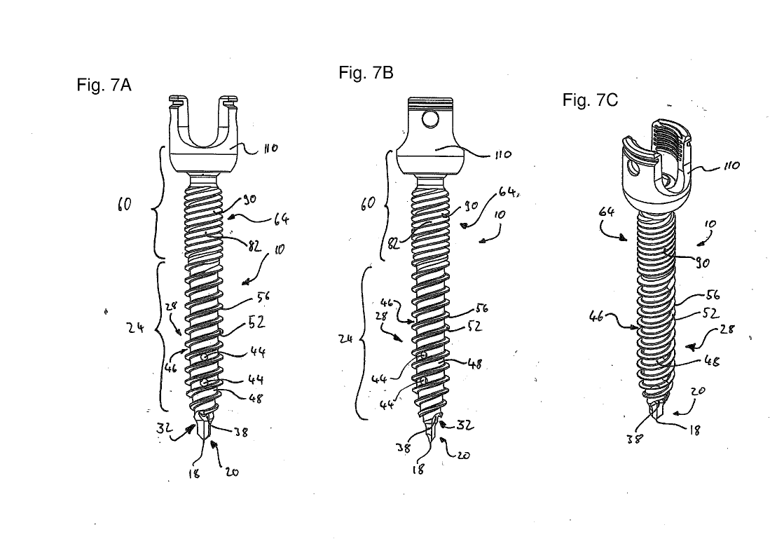

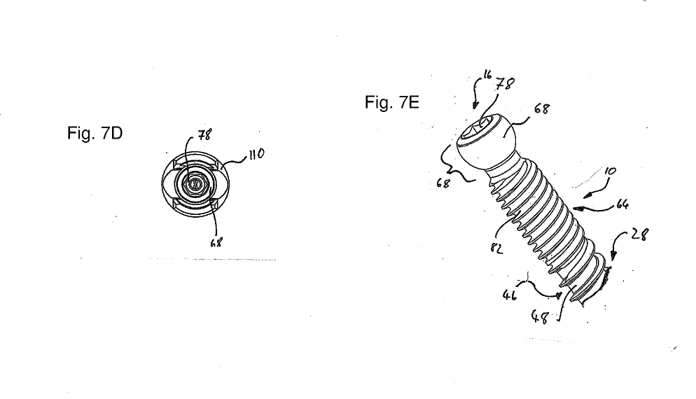

FIGS. 7A to 7D show a further embodiment of the bone screw according to the invention. The further embodiment according to FIGS. 7A to 7D comprises a receiving part 110, which serves to connect the bone screw 10 to a rod system 114.

In the present case, the receiving part 110 is connected to the bone screw 10 in a polyaxial manner. The term “polyaxial” is to be understood as a connection in which the receiving part 110 is pivotable and rotatable with respect to the rest of the bone screw 10. The polyaxial connection between the receiving part 110 and the rest of the bone screw 10 is realized by a screw head 68, which in particular has a dome-like shape on its underside, and by a corresponding and complementary mating surface on the receiving part 110. The dome-like screw head 68 is shown in FIG. 7E, which shows an area of the bone screw 10 shown in FIGS. 7A to 7D, without the receiving part 110.

In addition to a polyaxial connection, however, a uniplanar connection is also possible within the meaning of the invention. A uniplanar connection is to be understood as a connection in which the receiving part 110 is pivotable with respect to the rest of the bone screw 10 only within one plane. Within the meaning of the invention, a monoaxial connection is also possible, that is to say a connection in which the receiving part is not pivotable with respect to the rest of the bone screw. In the case of a monoaxial connection, the receiving part 110 can be rotatable with respect to the rest of the bone screw 10 or can be rigidly connected to the rest of the bone screw 10, for example formed in one piece with the latter.

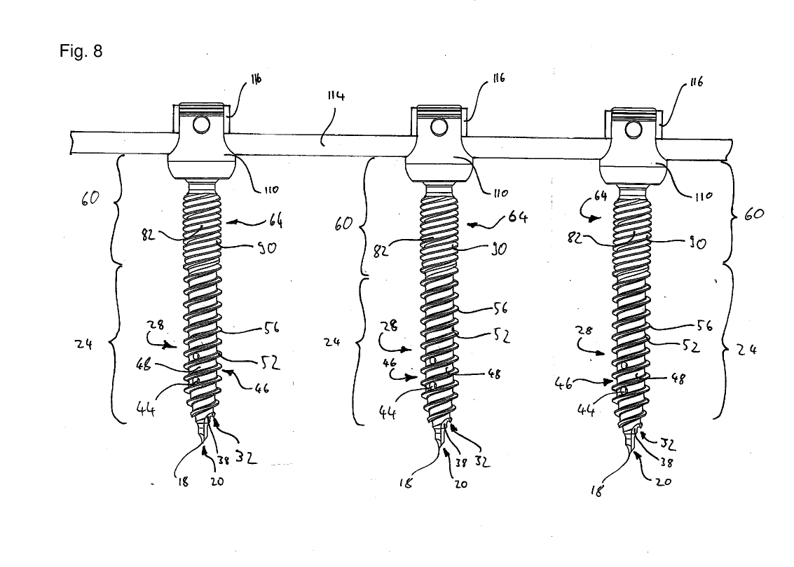

FIG. 8 shows three bone screws 10, which correspond to the bone screw 10 shown in FIGS. 7A to 7D and are connected to a rod system 114 via their respective receiving part 110. The rod system 114 is secured in the respective receiving parts 110 by a respective securing element 116 on the receiving part 110. Such a rod system 114 is used in surgical procedures for the treatment of spinal injuries.

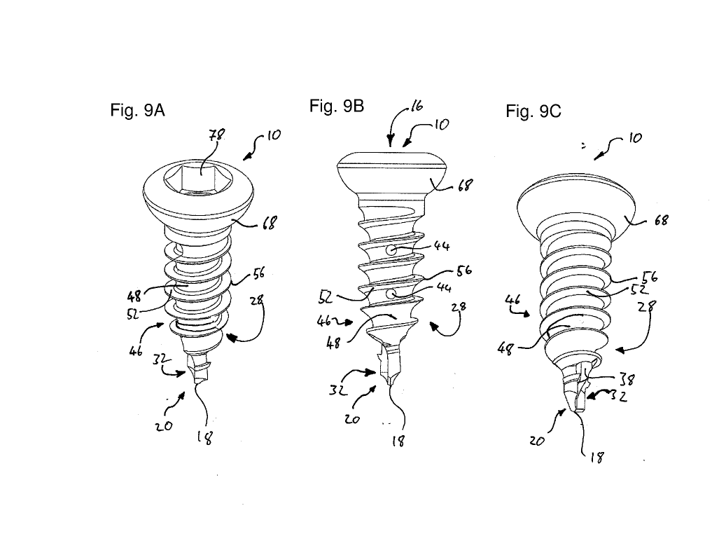

FIGS. 9A-C each show different views of a further embodiment of a bone screw 10 according to the invention. The bone screw 10 shown in FIGS. 9A-C has a screw head 68, which in particular has a dome-like configuration on its underside. This serves to secure the bone screw 10 at a variable angle on a bone plate, which has a corresponding and complementary receiving opening. The bone plate is not shown in the present case.

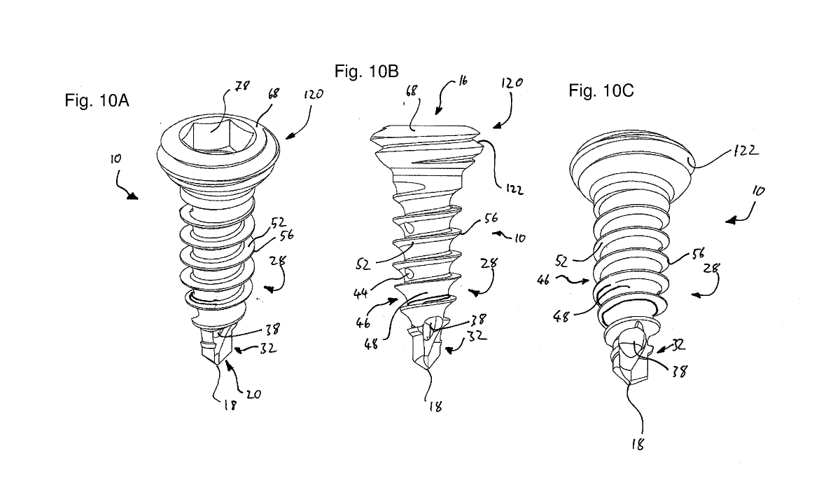

FIGS. 10A-C each show different views of a bone screw 10 according to the invention, comprising a screw head with a locking device 120. The locking device 120 is configured in the present case as a head-side outer thread 122. The locking device 120 serves to lock the bone screw 10 at a stable angle onto a bone plate (not shown). For said locking, the bone screw 10 is screwed with the head-side outer thread into an inner thread of complementary shape on an opening in the bone plate. This screwing-in produces an angularly stable connection between the bone plate and the bone screw 10.

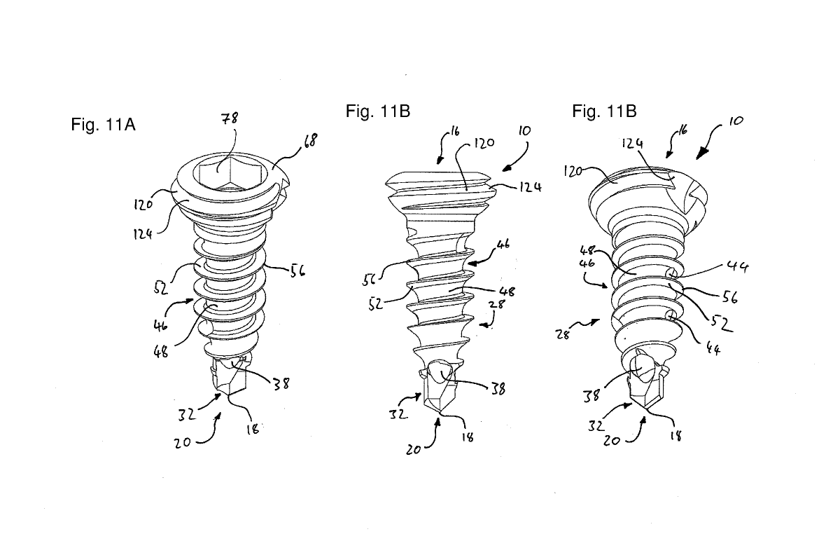

FIGS. 11A-C show different views of a further embodiment of the bone screw 10 according to the invention. The bone screw 10 shown in FIGS. 11A-C has, on its screw head 68, a self-cutting head-side outer thread 124. This self-cutting head-side outer thread 124 can be screwed into an opening in a bone plate, and, during the screwing-in procedure, a thread is cut into this opening of the bone plate. After the screwing-in procedure, the bone screw 10 is then locked at a stable angle in the bone plate.

1. A self-cutting and self-drilling cannulated bone screw (10) comprising a screw body with a channel chamber (12) defined therein, and a centering tip (20), which forms a front end (18) of the bone screw (10).

2. The bone screw (10) as claimed in claim 1, further comprising at least one cutting device (32, 34) arranged in an area of the centering tip (20).

3. The bone screw (10) as claimed in claim 2, wherein the cutting device (32, 34) includes an access opening (38, 40) to the channel chamber (12).

4. The bone screw as claimed in claim 1, further comprising at least two cutting devices (32, 34) arranged in an area of the centering tip (20) distributed about a circumference, and each of the cutting devices (32, 34) is assigned a substantially axial access opening (38, 40) to the channel chamber (12).

5. The bone screw (10) as claimed in claim 1, wherein the channel chamber (12) opens into a rear end face (19) of the bone screw (10).

6. The bone screw (10) as claimed in claim 1, wherein bone screw (10) is a Herbert screw.

7. The bone screw (10) as claimed in claim 1, further comprising a screw head (68) located at a rear end (16) of the bone screw (10), said screw head (68) has an abutment face (74) in the form of a radially extending collar (72).

8. The bone screw (10) as claimed in claim 1, further comprising at least one substantially radial opening (44) that extends from the channel chamber (12) to a thread turn (46) of an outer thread (28, 64) present in at least one of a front area (24) or a rear area (60) of the bone screw (10).

9. The bone screw (10) as claimed in claim 1, further comprising at least one substantially axial through-opening (86) in a flank (52, 90) of an outer thread (28, 64) present in at least one of a front area (24) or a rear area (60) of the bone screw (10).

10. The bone screw (10) as claimed in claim 9, wherein there are two of the through-openings (86), with one of the through opening being located in each of at least two successive ones of the flanks (52, 90) of at least one of the outer threads (28, 64), and the through-openings (86) are in alignment with each other.

11. The bone screw (10) as claimed in claim 1, further comprising at least one recess (94) in a protruding edge (56) of a flank (52) of a first outer thread (28, 64) present in a front area (24) of the bone screw (10), said recess (94) forms, at least in part, an additional cutting edge (98).

12. The bone screw (10) as claimed in claim 1, further comprising an outer thread (28, 64) which has at least one thread channel (102, 108) extending at least in part inside a flank (52) of the outer thread (28, 64) and extending at least in part in a circumferential direction, one end of said thread channel (102, 108) opening into an outer face of the outer thread (28, 64).

13. The bone screw (10) as claimed in claim 12, wherein the one end of the thread channel (102, 108) is arranged in an additional cutting edge (98) on the outer thread (28, 64) of the bone screw (10).

14. The bone screw (10) as claimed in claim 13, wherein the thread channel (102, 108) substantially follows a pitch of the outer thread (28, 64).

15. The bone screw (10) as claimed in claim 12, wherein the thread channel (102, 108) opens with its other end into the channel chamber (12) or into a next additional cutting edge (98), as seen in the circumferential direction, on the outer thread (28, 64).

16. The bone screw (10) as claimed in claim 1, further comprising a receiving part (110) for connecting the bone screw (10) to a rod system (114).

17. The bone screw (10) as claimed in claim 16, wherein the receiving part (110) is connected to the bone screw (10) in a polyaxial, uniplanar or monoaxial manner.

18. The bone screw (10) as claimed in claim 1, further comprising a screw head (68), which has a locking device (120) adapted to lock the bone screw (10) at a stable angle onto a bone plate.

19. The bone screw (10) as claimed in claim 1, further comprising a self-cutting head-side outer thread (124) adapted to lock the bone screw (10) at a stable angle onto a bone plate.

20. The bone screw (10) as claimed in claim 1, further comprising screw head (68) which in part has a dome-shaped configuration and via which the bone screw (10) is adapted to be connected at a variable angle to a bone plate.
